(19) United States
(12) Patent Application Publication
(10) Pub. No.: US 2005/0222013 A1
(43) Pub. Date: Oct. 6, 2005

(54) Methods for the use of inhibitors of histone deacetylase as synergistic agents in cancer therapy

(75) Inventors: Mira Jung, Rockville, MD (US); Manfred Jung, Gundelfingen (DE); Anatoly Dritschilo, Bethesda, MD (US)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: Georgetown University, Washington, DC (US)

(21) Appl. No.: 10/512,980

(22) PCT Filed: Jan. 16, 2004
(86) PCT No.: PCT/US04/01019

§ 371 Date: May 13, 2005

Related U.S. Application Data
(60) Provisional application No. 60/440,349, filed on Jan. 16, 2003.

(51) Int. Cl. A61K 38/12; A61K 31/19; A61N 5/00

(52) U.S. Cl. 514/9; 514/575; 600/1

(57) ABSTRACT

Improved methods for treatment of cancer are provided. The improvements include the administration of one or more synergistic agents, specifically inhibitors of histone deacetylase proteins and complexes. These synergistic agnets increase the effectiveness of radiation therapy and/or chemotherapies by increasing the sensitivity of tumor cells to treatment.

### BACKGROUND OF THE INVENTION

1. Field of the Invention

The invention relates to the use of inhibitors of histone de-acetylase as synergistic agents in improved cancer therapies.

2. Description of the Related Art

Radiotherapy, also called radiation therapy, is the treatment of cancer and other diseases with ionizing radiation. Ionizing radiation deposits energy that injures or destroys cells in an area being treated (a “target tissue”) by damaging their genetic material, making it impossible for these cells to continue to grow. Although radiation damages both cancer cells and normal cells, the latter are better able to repair themselves and function properly. Radiotherapy can be used to treat localized solid tumors, such as cancers of the skin, tongue, larynx, brain, breast, prostate, colon, uterus and/or cervix. It can also be used to treat leukemia and lymphoma (cancers of the blood-forming cells and lymphatic system, respectively).

One type of radiation therapy commonly used involves photons, “packets” of energy. X-rays were the first form of photon radiation to be used to treat cancer. Depending on the amount of energy they possess, the rays can be used to destroy cancer cells on the surface of or deeper in the body. The higher the energy of the x-ray beam, the deeper the x-rays can go into the target tissue.

Gamma rays are another form of photons used in radiotherapy. Gamma rays are produced spontaneously as certain elements (such as radium, uranium, and cobalt 60) release radiation as they decompose, or decay. Each element decays at a specific rate and gives off energy in the form of gamma rays and other particles. X-rays and gamma rays have the same effect on cancer cells.

A goal in radiation therapy is to uniformly radiate target tissue while minimizing the exposure of normal tissue. For this purpose, imaging and three-dimensional simulation coupled with immobilization of the body by various restraints has become important. Additional modes include stereotactic methods where multiple sources are simultaneously focused into a tissue volume from multiple angles.

Another technique for delivering radiation to cancer cells is to place radioactive implants directly in a tumor or body cavity. This is called internal radiotherapy. (Brachytherapy, interstitial irradiation, and intracavitary irradiation are types of internal radiotherapy.) Using internal radiotherapy, the radiation dose is concentrated in a small area, and the patient stays in the hospital for a few days. Internal radiotherapy is frequently used for cancers of the tongue, uterus, prostate, colon, and cervix.

Several new approaches to radiation therapy are being evaluated to determine their effectiveness in treating cancer. One such technique is intraoperative irradiation, in which a large dose of external radiation is directed at the tumor and surrounding tissue during surgery. Another investigational approach is particle beam radiation therapy. This type of therapy differs from photon radiotherapy in that it involves the use of fast-moving subatomic particles to treat localized cancers. Some particles (neutrons, pions, and heavy ions) deposit more energy along the path they take through tissue than do x-rays or gamma rays, thus causing more damage to the cells they hit. This type of radiation is often referred to as high linear energy transfer (high LET) radiation.

Other recent radiotherapy research has focused on the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells that are destroyed by the cell-killing (cytotoxic) action of the radiation. This approach can minimize the risk of radiation damage to healthy cells. The success of this technique will depend upon both the identification of appropriate radioactive substances and determination of the safe and effective dose of radiation that can be delivered in this way. Radiation therapy can be used alone or in combination with chemotherapy or surgery.

There is a need in the art for a means for increasing the effectiveness of radiation therapy and chemotherapy. In this regard, two types drugs are being studied for their effect on cells undergoing radiation. Radiosensitizers make tumor cells more susceptible to damage, and radioprotectors protect normal tissues from the effects of radiation. Radiosensitizers in current trials include cisplatin and 5-fluorouracil as well as antibodies directed against growth factors.

### SUMMARY OF THE INVENTION

The present invention relates to a treatment for cancer that combines the administration of a synergistically effective amount of at least one inhibitor of histone deacetylase with at least one other anticancer treatment. More specifically, this invention relates to a treatment for cancer comprising administering a synergistically effective amount of at least one inhibitor of histone deacetylase and conducting radiation therapy. Alternatively, this invention relates to a treatment for cancer comprising administering a synergistically effective amount of at least one inhibitor of histone deacetylase and administering an effective amount of at least one other anticancer drug.

Preferably, the histone deacetylase inhibitor is a reversible inhibitor and is administered for a period prior to and/or during the administration of radiation and/or chemotherapy, and optionally continuing for a period after radiation and/or chemotherapy. More preferably, the histone deacetylase inhibitor is chosen from among the compounds selected from the group consisting of trichostatin A, FR, M344, SAHA, combinations thereof, and the like. Alternatively, the histone deacetylase inhibitor can be selected from the group consisting of the compounds listed in Table 1, combinations thereof, and the like.

Preferably, the amount of histone deacetylase inhibitor administered is sufficient to produce a concentration of the inhibitor in a target tissue site that is effective in synergistically enhancing the primary radiation or chemotherapy treatment and low enough to avoid systemic toxicity to the host. More preferably, the amount of inhibitor administered is sufficient to produce a concentration of the inhibitor at the target tissue that is about equal to or less than the IC₅₀.

### BRIEF DESCRIPTION OF THE DRAWINGS

The objects and advantages of the invention will become apparent from the following detailed description of the preferred embodiments thereof in connection with the accompanying drawings, in which:

FIG. 1 shows effects of the HDAC inhibitor trichostatin A (TSA) on clonogenic survival of SQ-20B in response to ionizing radiation. Logarithmically growing SQ-20B cells were treated with TSA (about 60 ng/ml) for about 24 h and the medium was replaced with fresh medium. Cells were exposed to a graded dose of gamma radiation. A semi-logarithmic plot of the data for these cells is shown. Clonogenic survival numbers were determined and fit to the single hit multitarget and the linear quadratic models for analysis, measured by clonogenic survival analysis. Points and bars represent mean+/−SEM from triplicate flasks in each experiment.

FIGS. 2A and 2B show cell cycle distributions of SQ20B cells after treatment with either TSA (FIG. 2A) or mock treatment (FIG. 2B) for 24 h. Cells were washed with fresh medium and exposed to about 10 Gy ionizing radiation. Cell nuclei were prepared for flow cytometric analysis using the procedure of Vindelov et al. (46). The samples were analyzed on a Becton-Dickinson FACStarplus instrument and the percentage of nuclei with G1, S, and G2/M DNA content was determined.

FIG. 3 shows the relative numbers of SQ-20B cells in G1 phase following about 24 hour exposure to Mimosine (about 0.4 mM), TSA (about 60 ng/ml) or irradiation (about 10 Gy).

FIG. 4 shows a cell growth analysis. Following treatment with either TSA (about 60 ng/ml) or mock, cells were seeded and maintained in the presence or absence of TSA and counted at various intervals by using tryphan blue exclusion method.

FIG. 5 shows effects of irradiation on the apoptotic index in SQ-20B cells pretreated with TSA or mock treatment. SQ-20B cells were exposed to about 10 Gy of irradiation. Time zero refers to cells that were subjected to sham irradiation. At the indicated times thereafter, attached and floating cells were collected and the number of apoptotic cells was determined as a percentage of the total number of cells (apoptotic index). Data shown represent mean values±SD from three independent experiments.

FIG. 6 shows exemplary HDAC inhibitor compounds.

### DETAILED DESCRIPTION OF THE PREFERRED EMBODIMENTS OF THE INVENTION

DNA in the eukaryotic nucleus is packaged into highly organized chromatin. One function of chromatin packaging is to control access to promoters for DNA-binding proteins that regulate transcription. In the process of activation or repression of gene expression the chromosomal structure undergoes extensive remodeling. Mechanisms for altering chromosomal structure include post-translational modifications of histones and adenosine-triphosphate(ATP)-dependent chromosome remodeling. These two processes appear to work in concert to achieve precise control of gene expression by extensive post-translational modifications, such as acetylation, deacetylation, phosphorylation, and methylation.

Acetylation at lysine residues on the amino-terminal tails of histones neutralizes the positive charged residues and decreases the affinity of the histones for DNA. This results in a less constrained packaging of DNA. Repair proteins are thus provided with increased access to DNA damage and transcriptional regulatory proteins gain access to chromatin templates. Conversely, the hydrolysis of acetyl groups, deacetylation, restores a positive charge thereby providing for higher order chromatin folding.

Histone acetyltransferase (HAT) proteins were previously identified as having functions in transcriptional regulation. For example, TAF130/250 HAT is a subunit of the TFIID complex that is a component of the Pol II transcriptional machinery. Likewise, the p300/CBP HAT was initially described as a transcriptional co-activator that functioned by interacting with various enhancer binding proteins. The p300 protein also acetylates p53 at the C-terminal DNA binding domain and interacts with other factors.

Currently, eleven mammalian histone deacetylase (HDAC) enzymes have been identified, which are homologs of either RPD3 or HDA1 yeast histone deacetylases. HDACs are components of large complexes in both mammalian and yeast cells with varied functionality. Inhibition of HDACs with trichostatin A (TSA) has been observed to have effects at specific promoters. The HDAC1 interacts with the Rb-binding protein RbA-p48, which can physically link Rb with HDACs. HDAC2 interacts with ATR and two different families of transcriptional repressors: nuclear hormone receptors and Mad, an antagonist of Myc. HDAC4 is associated with MAK kinase activity.

Recent studies revealed an additional family of cellular factors that possesses intrinsic HAT or HDAC activities. These appear to be non-histone proteins that participate in regulation of the cell cycle, DNA repair, and transcription. A number of transcriptional coactivators, including p400AF, BRCA2, and ATM-like protines, function as HAT's. Some transcriptional repressors exhibit HDAC activities in the context of chromatin by recruiting a common chromatin-modifying complex. For instance, the Mas protein family (Mas1, Mxi1, Mad3, Mad4) comprises a basic-helix-loop-helix-loop-helix-zipper class of transcriptional factors that heterodimerize with Max at their DNA binding sites. Mad:Max heterodimers act as transcriptional repressors at their DNA binding sites through recruitment of “repressor complexes.” Mutations that prevent interaction with either Max or the msin3 corepressor complex fail to arrest cell growth.

One mode of cellular regulation arises from the ability of HDAC proteins to bind to pRB/E2F/DP complexes. The intrinsic deacetylases of this complex appear to contribute to down-regulation of genes by the pRb family of proteins. Rb serves as a physical bridge, tethering the activity of HDAC1 to E2F and, by association, to E2F promoters. Repression of E2F-bound promoters by Rb is considered to be one of the mechanisms by which Rb induces growth arrest. Rb proteins, phosphorylated in the G1 phase, play a major role as negative regulators of cell progression toward the S phase. HDAC1 preferentially binds to the active, hypophosphorylated, form of Rb which leads to the release of free E2F. Release of free E2F leads in turn to the activation of transcription of its target genes. E2F1 is observed to interact with CBP which also possesses HAT activity. Taken together, these observations suggest that a deactivating complex having HAT activity functions by displacing the E2F1/Rb/HDAC1 repressor complex. From the above, and other data, it can be understood that the equilibrium between HAT's and HDAC's is critical for proper cell cycle control.

The increased effectiveness of radiation therapy provided by the present invention is believed to arise, at least in part, because de-condensed chromatin appears to be more sensitive to radiation damage than condensed chromatin. It is commonly understood that histone acetylation allows critical proteins to access DNA for repair and screening for DNA damage. However, where the regulation of acetylation states is disrupted, cells also become vulnerable to genotoxic damage. In the present invention, without wishing to be bound by theory, it is believed that HDAC inhibitors function to disrupt the equilibrium of acetylation states and thereby increase cell killing by ionizing radiation and chemotherapy.

Following exposure of cell to radiation, a number of molecules are activated in response to DNA damage. Arrest of cell cycle progression, transcription of specific genes, and activation of the DNA repair machinery is observed. For example, the ATM gene, which is mutated in the human disease ataxia telangiectasia, is directly linked to extreme cellular radiation sensitivity. ATM interacts with HDAC1 and the complex exhibits HDAC activity. These observations provide support for HDAC activity and chromatin modification in the mechanism associated with intrinsic cellular radiation sensitivity. See, for example, reference 44.

On the basis of these and other observations, we have discovered that histone deacetylase (HDAC) inhibitors can be employed in conjunction with radiation treatment of neoplastic disorders such as various cancers to provide a synergistic effect. A HDAC inhibitor is a compound or composition that inhibits one or more HDAC proteins (i.e. HDAC1, HDAC2, HDAC3, HDAC4, and the like) or any other biological protein or complex with histone deacetylase activity. A HDAC inhibitor for use in the present methods can be a general inhibitor of a plurality of HDAC active proteins. Alternatively, the inhibitor can be specific for one or more than one enzyme. Because of the apparent participation of various HDAC proteins in specific control pathways, an additional advantage can be realized by choosing an inhibitor that interferes with radiation survival of a specific neoplastic cell type. The relative effectiveness of any particular HDAC inhibitor in a given tumor type can be assessed by measuring the radiation survival of cultured cells from a similar tumor type. Alternatively, and more specifically, the relative effects of any HDAC inhibitor can be determined by analysis of the transcriptional profiles of treated and untreated cells. By this method, the effect of treatment on specific transcriptional control pathways can be determined and selected for optimum advantage.

The present invention provides methods of treating cancers comprising the administration of an effective amount of a HDAC inhibitor as a synergistic agent in conjunction with recognized methods of radiotherapy and chemotherapies, including, for example, chemical-based mimics of radiation therapy whereby a synergistic enhancement of the effectiveness of the recognized therapy is achieved. A synergistic effect, as provided by the present methods, means a statistically significant increase in the effectiveness of a conventional treatment, such as a radiotherapy or chemotherapeutic treatment, particularly, where a treatment with HDAC inhibitor alone, at the dosage used in the combination treatment, would not provide as great a therapeutic effect. The effectiveness of a treatment may be measured in clinical studies or in model systems, such as a tumor model in mice, or cell culture sensitivity assays.

The present invention provides novel strategies for combination therapies that result in improved effectiveness and/or reduced toxicity. According to one aspect of the invention, HDAC inhibitors are employed as radiosensitizers in conjunction with radiotherapy. Preferred dosages and administration regimes can further provide improved effectiveness. It is a further aspect of the invention that HDAC inhibitors can be administered in conjunction with chemotherapies to provide synergistic effects. In a preferred aspect of the invention, HDAC inhibitors can be used in combination therapy with chemical agents that are understood to mimic the effects of radiotherapy and/or that function by direct contact with DNA, such as, for example, DNA alkylating agents. Preferred agents for use in combination with HDAC inhibitors in methods according to the invention include cisplatinum, adriamycin (Doxirubicin), topoisomerase inhibitors (Etoposide), 5-FU, and taxol.

Prior approaches to the use of HDAC inhibitors in therapeutic compositions have typically employed the maximum tolerated dose. However, the maximum tolerated dose can produce undesirable effects. We have surprisingly discovered that the concentration of HDAC inhibitor necessary to provide a synergistic effect, such as radiation sensitization, is significantly less than the concentration necessary to cause tumor cell death as a single agent, such as by apoptosis. It is therefore, a further aspect of the invention that because of the synergistic effectiveness of HDAC inhibitor compounds, the compounds of the invention can be effectively used in combination therapy methods at dosages that are substantially less than dosages used in single agent applications. According to this aspect of the invention, HDAC inhibitors can be used synergistically at effective amounts that result in concentrations in the fluid of a target tissue that are less than about twice the IC₅₀ concentration for the particular compound, more preferably about equal to the IC₅₀ concentration. Alternatively, the HDAC inhibitors may be administered at lower amounts such as about 50% of the IC₅₀ concentration, or less, at the target tissue. Furthermore, the HDAC inhibitor can be administered locally so that the concentration at the target tissue is in the effective range and lower elsewhere.

IC₅₀ is defined as the concentration of the HDAC inhibitor that kills 50% of cells following treatment with the drug. To determine the IC₅₀, a series of dilutions of drug is used over a broad range, for example 0-1.6 μg/ml. Approximately 100-400 cells are seeded into T-25 flasks in triplicate and treated with various concentrations of the drug. After a 24 hour treatment, the cells are washed with standard PBS (phosphate buffered saline) solution. Cells are then grown in fresh media for 2 to 3 weeks. Colonies are stained with crystal violet and scored. Concentrations of drugs showing 50% cell death represent the IC₅₀ concentration.

HDAC inhibitors are known to promote cancer cell death through apoptosis in vitro and in vivo. This has been a basis for the use of HDAC inhibitors at high concentrations as a single agent in chemical therapy. However, we have determined that the amount effective to synergize radiation-induced cell death is substantially less than the amount required to promote apoptosis. In a preferred aspect of the invention, the optimal dosage of HDAC inhibitor results in a concentration at a target tissue that does not promote apoptosis of cells in culture yet is effective in increasing cell death in neoplastic cells exposed to radiation or recognized chemotherapeutic chemical agents. For example, TSA did not promote significant apoptosis at the IC₅₀ concentration of about 60 ng/ml but did promote significant apoptosis at concentrations of about 200-about 400 ng/ml. Administration of TSA at about the IC₅₀ concentration significantly sensitized head and neck tumor cells to ionizing radiation. Concentrations that produce these effects can be determined for any HDAC inhibitory compound by one of skill in the art by observation of markers of apoptosis such as, for example, the apoptotic index and caspase activities.

In another aspect of the invention, HDAC inhibitors are administered one or more times a day during the course of radiation or chemical therapy. It may be desirable to not administer either the HDAC inhibitors or the radiation or chemical therapy on certain days during the treatment period. For example, according to the method of the invention, treatment can be administered for approximately four to six weeks except on every sixth day, or a similar schedule. In an alternative regimen, the HDAC inhibitors are may administered during the first and last portions of the radiation and/or chemical treatment. For example, the HDAC inhibitors can be administered during approximately the first and last quarters of the radiation or chemical treatment period or the first and last third of the radiation or chemical treatment period, such as for the first and last two weeks of a total six-week treatment period. In addition, the HDAC inhibitors can be administered for any period of, for example, 0 to about 14 days prior to treatment. The preferred dose is chosen to sustain a concentration at the target tissue, as described above, for the period of treatment. In a preferred aspect of the invention, an HDAC inhibitor of the invention is administered at the prescribed dose about 10 times over about 13 days prior to the start of radiation treatment. The administration of an HDAC inhibitor may then be continued during the period of radiation treatment.

HDAC inhibitors can be administered as synergistic agents in the form of pharmaceutically acceptable salts. One or more synergistic agents can be used in a combination therapy. Any such pharmaceutically acceptable salts can be used so long as they do not adversely affect the desired pharmacological effects of the HDAC inhibitors. Selection and production of a composition in accordance with the invention can be made by those skilled in the art. For example, as a pharmaceutically acceptable salt, an alkali metal salt such as a sodium salt or a potassium salt, an alkaline earth metal salt such as calcium salt or a magnesium salt, a salt with an organic base such as an ammonium salt, or a salt with an organic base such as a triethylamine salt or an ethanolamine salt, can be used. Subjects to be treated by the present invention include both humans and animals.

The synergistic agents of the present invention can be administered orally or non-orally. In the case of oral administration, an agent can be administered in various suitable forms. Suitable forms include, but are not limited to, soft and hard capsules, tablets, granules, powders, solutions, suspensions, combinations thereof and the like. In the case of non-oral administration, they can also be administered in a variety of suitable forms. Suitable forms include, but are not limited to, ointments or injection solutions, drip infusion formulations, suppositories whereby continuous membrane absorption can be maintained in the form of solid, viscous liquids, suspensions, combinations thereof and the like. The selection of the method for preparation of these formulations and the vehicles, disintegrators or suspending agents, can be readily made by those skilled in the art. The synergistic agent of the present invention can include a further substance having radiosensitizer activity in addition to HDAC inhibitors or their pharmaceutically acceptable salts.

In an alternative embodiment of the invention, the HDAC inhibitor can be administered locally at a target tissue, such as by injection, infusion, or by implantation of an arrangement adapted to release the compound at a controlled rate over a period of time. Such an arrangement might comprise, for example, an osmotic pump arrangement. The arrangement might also include a radiation source such as, for example, a radiation source suitable for internal radiation therapy.

In alternative embodiments of the invention, the HDAC inhibitory compound can be administered in encapsulated form in a vehicle adapted to deliver the compound preferentially to the target tissue such as, for example, by targeted liposomes or the like. By targeting delivery to the affected tissue, an effective concentration can be administered while systemic effects are minimized.

The amount of the active ingredients in the pharmaceutical composition of the present invention can vary depending on the formulation, but will usually be in the range from about 0.1 to about 50% by weight, regardless of the manner of administration. The individual dose will be determined according to the principles set forth herein taking into consideration the age, sex, and symptoms of the disease of the subject, the desired effect, the period for administration, etc. Practitioners in the art are able to formulate a dosage and administration regimen that will achieve a desired concentration at the treatment site.

Any inhibitor of HDAC activity that provides a synergistic effect in combination with radiotherapy or chemotherapy can be used in accordance with the principles of the invention, provided that the inhibitor has acceptably low toxicity to the host. The toxicity of any prospective compound can be routinely determined by one of skill in the art. However, the following are preferred characteristics of the HDAC inhibitory synergistic agent of the invention: high inhibitory activity at low concentrations (preferably having an IC₅₀ of less than about 800 ng/ml, more preferably about 320 ng/ml or less or most preferably about 60 ng/ml or less, i.e. about 5 ng/ml), reversible HDAC inhibition, low toxicity at synergistic doses, rapid clearance following termination of administration. An acceptable combination of these characteristics can include compromises in one or more categories, however the advantages of the invention are best achieved in the combination of these characteristics.

The effectiveness of a particular treatment can be determined by measuring the radiation survival. Post treatment survival can be studied both in vitro and in vivo. For example, for in vitro determinations, exponentially growing cells can be exposed to known doses of radiation and the survival of the cells monitored. Irradiated cells are plated and cultured for about 14-about 21 days, and the colonies are stained. The surviving fraction is the number of colonies divided by the plating efficiency of unirradiated cells. Graphing the surviving fraction on a log scale versus the absorbed dose on a linear scale generates a survival curve. Survival curves generally show an exponential decrease in the fraction of surviving cells at higher radiation doses after an initial shoulder region in which the dose is sublethal. A similar protocol can be used for chemical agents.

Inherent radiosensitivity of tumor cells and environmental influences, such as hypoxia and host immunity, can be further assessed by in vivo studies. The growth delay assay is commonly used. This assay measures the time interval required for a tumor exposed to radiation to regrow to a specified volume. The dose required to control about 50% of tumors is determined by the TCD50 assay. In vivo assay systems typically use transplantable solid tumor systems in experimental animals. Radiation survival parameters for normal tissues as well as for tumors can be assayed by in vivo methods.

Two mathematical models are commonly employed to analyze radiation survival data. A first model is the multi-target model. In this analysis, the reciprocal of the slope of the survival curve is defined as D₀, the radiosensitivity of the cell population or tissue under investigation. D₀ is the dose required to reduce the surviving fraction to about 37% in the exponential portion of the survival curve. The extrapolation of the linear portion of the curve to the y-intercept is denoted n. The width of the shoulder region is represented by drawing a line from the 100% survival point to the extrapolation line, this width is denoted D_{q}. D_{q} is the quasi-threshold dose, or the point at which the reduction in surviving fraction as a function of radiation dosage becomes exponential. The D_{q} value can also provide an estimate of an additional total dose required for each division of a single dose therapy into fractional doses. The additional dose is required to overcome the effect of sublethal damage repair that occurs when two sublethal doses are separated in time.

The linear quadratic model (surviving fraction=e^{αD-βD2}) is used to fit radiation survival data to a continuously bending curve, where D is dose and α and β are constants. Alpha is the linear component, a measure of the initial slope that represents single-hit killing kinetics and dominates the radiation response at low doses. Beta is the quadratic component of cell killing, that represents multiple-hit killing and causes the curve to bend at higher doses. The alpha:beta ratio is the dose at which the linear and quadratic components of cell killing are equal. The more linear the response to killing of cells at low radiation dose, the higher is the value of alpha, and the greater is the radiosensitivity of the cells.

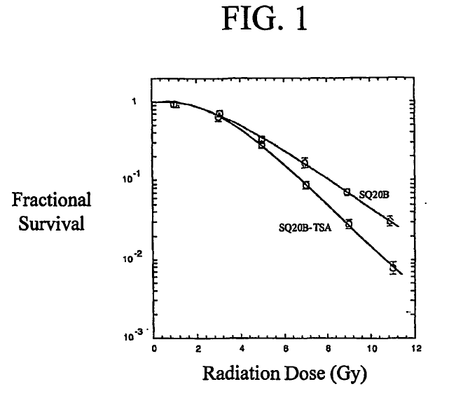

Referring to FIG. 1, the effects of TSA on clonogenic survival of SQ-20B cells in response to ionizing radiation is demonstrated. A semi-logarithmic plot of the mean+/−SEM from triplicate flasks in each experiment for these cells is shown. Logarithmically growing SQ-20B cells were treated with TSA (about 60 ng/ml) for about 24 h and the medium was replaced with fresh medium. Cells were exposed to a graded dose of gamma radiation. Clonogenic survivals were determined and fit to the single hit multitarget and the linear quadratic models for analysis. The IC₅₀ of TSA is about 60 ng/ml, these results demonstrate that a synergistic effect is produced at concentrations at least about equal to the IC₅₀ of the HDAC inhibitory compounds.

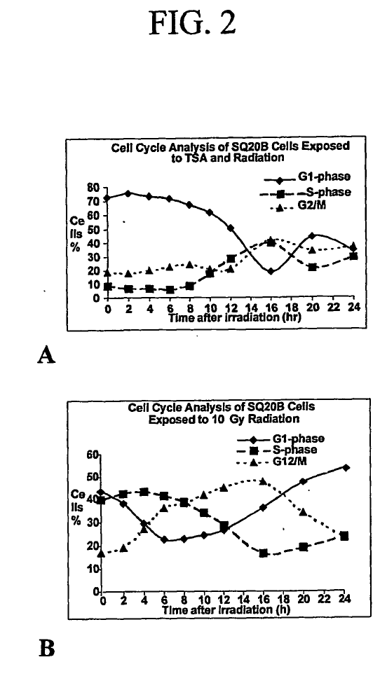

The radiation sensitivity of cells is a function of the cell cycle. The lethal effect of radiation exposure is often observed only after subsequent cell division. Accordingly, the G1 phase is the more radiation sensitive phase. The compounds employed in the invention are observed to maintain cells in the G1 phase following radiation exposure. Comparing FIG. 2A to FIG. 2B, it can be seen that exposure of SQ-20B cells to TSA for about 24 hours prior to radiation results in an accumulation of more than about 70% of cells in the G1 phase and a maintenance of about 50% of cells for about 12 hours. After treatment with either TSA (panel A) or mock treatment (panel B) for about 24 h, cells were washed with fresh medium and exposed to about 10 Gy ionizing radiation. Cell nuclei were prepared for flow cytometric analysis. The samples were analyzed on a Becton-Dickinson FACStarplus® instrument and the percentage of nuclei with G1, S, and G2/M DNA content was determined. By comparison, irradiated mock-treated cells are not arrested.

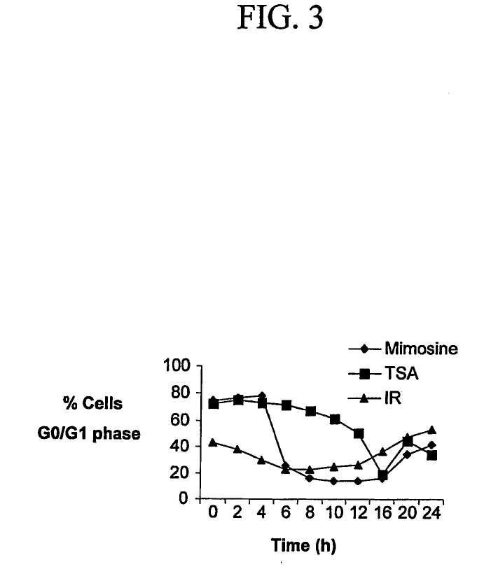

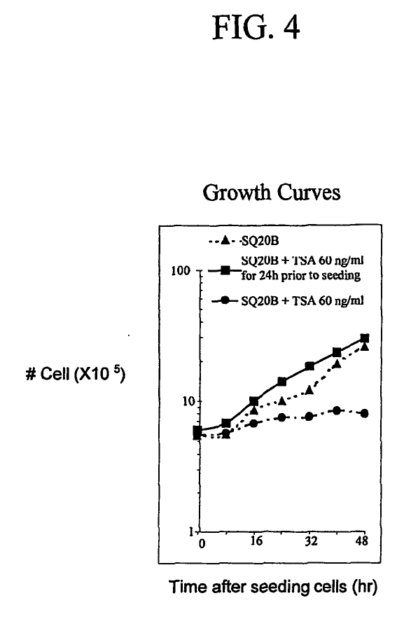

With reference to FIG. 3, which shows a comparison of the relative number of SQ-20B cells in G1 phase following about 24 hour exposure to mimosine (about 0.4 mM), TSA about 60 ng/ml or radiation (about 10 Gy), it is observed that the G1 arresting effect of pre-irradiation treatment with the HDAC inhibitor TSA is longer lasting (about 12 hours) than mimosine (about 4 hours), a known G1 synchronizing agent. Moreover, FIG. 4, shows a cell growth analysis following treatment with TSA (about 60 ng/ml) for about 24 hours, in the continued presence of TSA (about 60 ng/ml), or a mock treatment. This demonstrates that full recovery of cell growth is achieved following discontinuation of HDAC inhibitor exposure.

Current usage of HDAC inhibitors as single agents in cancer therapy is based on their function as potent inducers of apoptotic cell death. Because apoptotic death is known to be associated with cell cycle control, we originally hypothesized that the synergistic effect of HDAC inhibitors was a function of induction of apoptosis.

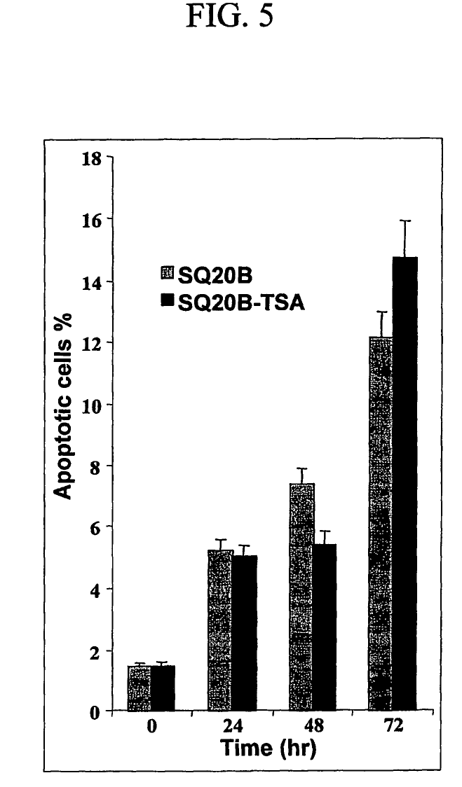

However, our data suggests that the synergistic radiation sensitization effect observed at lower concentrations is due to enhanced mitotic cell death rather than induced apoptosis. Treatment with TSA (about 60 ng/ml) or mock treatment was followed by radiation (about 10 Gy). At about 24, about 48, and about 72 hours, attached and floating cells were collected and the number of apoptotic cells was determined as a percentage of the total number of cells (apoptotic index). As shown in FIG. 5, surprisingly, it was found that at concentrations near the IC₅₀ (i.e., about 60 ng/ml TSA), the number of treated and untreated apoptotic cells were similar. At concentrations of about 3 to about 7 times the IC₅₀, TSA does promote apoptosis.

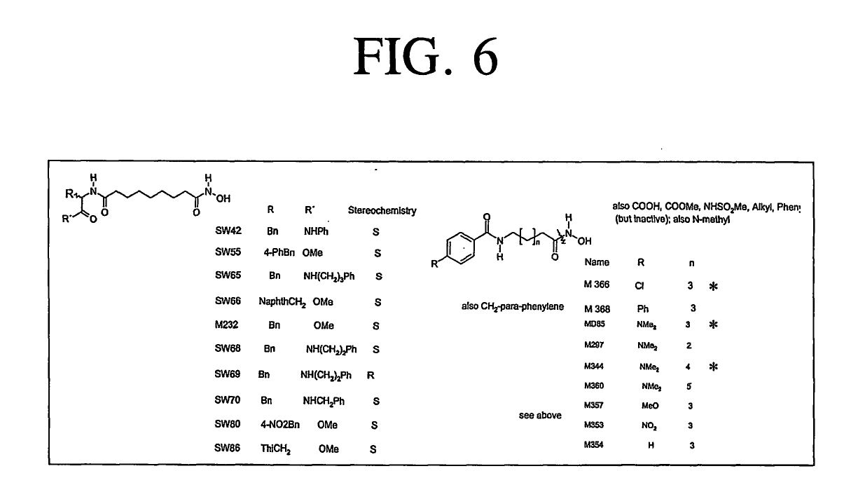

Referring to FIG. 6, additional examples of HDAC inhibitor compounds contemplated for use in the methods of the invention are shown. From in vitro radiation sensitization screening data, as shown in Table 1, three most preferred compounds are identified for use in the methods of the invention, FR, M344, and SAHA.

TABLE 1
Radiation Sensitizing Effects of HDAC Inhibitors at IC₅₀

| Cells-Drug | IC₅₀ (ng/ml) | α | β | Dₒ |
|---|---|---|---|---|
| SQ-20B | | 0.148 | 0.017 | 2.36 |
| SQ-20B-TSA | 60 | 0.126 | 0.030 | 1.64 |
| SQ-20B-FR | 5 | 0.104 | 0.033 | 1.65 |
| SQ-20B-M344 | 320 | 0.077 | 0.033 | 1.65 |
| SQ-20B-M366 | 70 | 0.106 | 0.014 | 2.70 |
| SQ-20B-MD85 | 100 | 0.024 | 0.022 | 2.49 |
| SQ-20B-SW14 | 380 | 0.057 | 0.019 | 2.48 |
| SQ-20B-H88 | 320 | 0.002 | 0.021 | 2.51 |
| SQ-20B-M293 | 800 | 0.083 | 0.020 | 2.30 |
| SQ-20B-M355 | 100 | 0.131 | 0.017 | 2.32 |
| SQ-20B-SAHA | 800 | 0.011 | 0.031 | 1.88 |

Note:
TSA: Tricostatin A
FR: depsipsptide (FR 901228)
SAHA: suberonylanilide hydroxamic acid
M & MD: amide analogues of Trichostatin A
SW: hydroxyamic acid analogues of Trapoxin
H: Scriptaid analogues

### EXAMPLES

The efficacy of any particular embodiment of the method of treatment can be assessed in a murine model of tumor growth and treatment.

Measurement of pharmacokinetics and toxicity of drugs. To evaluate in vivo introduction of HDAC chemical inhibitors, test drugs are injected intravenously, for example, via the tail vein into tumor bearing Balb/c nu/nu mice. Control mice are injected with normal saline. Blood is collected in heparinized tables at about 5 min, about 15 min, about 30 min, about 1 h, about 2 h, about 4 h, about 8 h, about 24 h and about 48 h after drug administration. Five mice are tested per time point. Mice are then euthanized and liver, spleen, kidney, lung, heart and tumor tissue are rapidly excised, rinsed in ice-cold normal saline and snap frozen on dry-ice. Blood samples are centrifuged at about 3000 rpm for about 10 min at about 40° C. to separate the plasma. The plasma and tissue samples are stored at about −800° C. for further analysis.

1) Plasma pharmacokinetic parameters are assessed by standard methods. The elimination rate constant (β) is calculated using the linear regression analysis of plasma concentration-time curve. The area under the curve (AUC) is calculated using the linear trapezoidal method with extrapolation of the terminal phase to infinity (Cₗₐₛₜ/β), where Cₗₐₛₜ is the last measured concentration. Other parameters are calculated as follows: Total body clearance (Cl)=Dose/AUC; volume of distribution (Vₐᵣₑₐ)=Cl/β; elimination half-life (t½β)=0.693/β.

2) Toxicity of drugs: Based on published data and our experience, it appears that deposition of drugs occurs not only in the human tumor xenografts, but also in normal tissue in mice. Mice are weighed and observed daily. Moribund mice are sacrificed and complete blood chemistry and histopathology is performed.

Tumor model in mice. Logarithmically growing SQ-20B tumor cells (about 2×10⁶) are injected subcutaneously in the lower back above the tail of Balb/c nu/nu mice. When palpable tumors grow (mean tumor volume of about 100 mm³), mice are divided into various treatment groups.

1) The effects of HDAC inhibitors in mice: Based on our experience with other radiation sensitizing drugs, administration of the selected dose in about 10 injections over about 13 days is optimal. The dosage schedule is tested for its ability to achieve inhibition of HDAC in vivo. Ten to fifteen animals are used at each dose level. At the end of drug treatment, tumors are excised and tissues are processed for histone and HDAC protein levels. In a parallel experiment, we determine the effect of drug treatment on tumor response. Tumor volumes are determined by caliper measurements of the three major axes (a,b,c) and calculated using abc/2, an approximation for the volume of an ellipse (πbc/6). Tumor volumes are monitored at least twice weekly.

2) Radiation of tumors: Tumors are grown in Balb/c nu/nu mice as described above. For radiation of tumors, animals are secured in a lead fabricated restraint that permits only the tumor area to be exposed to γ-irradiation. The tumors are irradiated using a [¹³⁷Cs] irradiator (J. L. Shepard Mark I). For SQ-20B tumors, a cumulative dose of about 40-about 50 Gy is used. These doses are based on our previous experience with these tumors in mice. Tumor volumes are measured as described above and radiation response for the tumors are determined. A similar protocol is used to calibrate the effectiveness of single chemotherapeutic agents. Chemotherapeutic agents are administered to mice using a dosage regimen consistent with its use as a single agent and the response of tumors is measured as described above.

3) Combination treatments of HDAC inhibitors with radiation or chemotherapy in tumor bearing mice: Based on the above experiments using single agents (HDAC inhibitors or radiation or chemotherapy agents) optimal combinations of drug and radiation doses are determined to achieve radiosensitization. A dose and treatment schedule of HDAC inhibitors resulting in no toxicity, tumor regression or inhibition of HDAC protein expression is selected for use in combination with radiation. A decrease in the relative tumor volumes in the combination-treated group as compared to single-agent-treated groups demonstrates synergistic improvement in radiosenstization. Similarly, chemotherapeutic agents are administered in combination with a HDAC inhibitor and the relative response of tumors is measured. Further controls include untreated and normal saline treated groups.

4) Statistical analysis of tumor growth: Tumor volumes are calculated as the percentage of pre-treatment tumor volume and the mean % tumor volume±S.E. are plotted. Analysis of variance (one-way ANOVA) statistical analysis is performed to demonstrate statistical significance of changes in tumor volumes. For multiple comparisons, Duncan's multiple range test is used.

5) Histopathology of tumor and normal tissue samples: tumor and normal tissues are obtained from treated and untreated animals, fixed in about 10% buffered formalin, blocked in paraffin, sectioned and stained with hematoxylin and eosin for histopathological examination.

Histones in tumor tissue and normal tissue are examined about 24 h after the last treatment with drug. The effects of HDAC inhibitors in tissue samples are examined by performing Western blotting with anti-acetyl histone antibodies and biochemical assays for apoptosis.

### REFERENCES

The following literature provides further background and teachings which may be of value to one of skill in the art in the practice of the invention.
1. Kornberg R and Lorch Y., Twenty-five years of the Nucleosome, Fundamental Particle of the Eukaryote Chromosome, Cell Press 98:285-294, 1999.
2. Bjorklund S, Almouzni G, Davison R, Nightingale K, Weiss K. Global Transcription Regulators of Eukaryotes. Cell. 96: 759-767, 1999.
3. Struhl K and Moqtaderi Z. The TAFs in the HAT. Cell. 94:1-4. 1998.
4. Oleinick N L, Balasubramaniam U, Xue L, and Chiu S. Nuclear structure and the microdistribution of radiation damage in DNA. Int J Radiat Biol 66:523-529, 1994.
5. Peters S J, Withers H R, Thames H E Jr. et al. Keynote address—The problem: tumor radioresistance in clinical radiotherapy. Int. J. Radiat. Oncol. Biol. Invest. 8:111-118, 1982.
6. Weichselbaum R R, Beckett M A, Dahlberg W, Dritschilo A. Heterogeneity of radiation response of a parent human epidermoid carcinoma cell line and four clones. Int J Radiat Oncol Biol Phys 14:907-912, 1988.
7. Meyn S M. Ataxia telangiectasia and cellular responses to DNA damage. Cancer Res. 55:5991-6001, 1995.
8. Jackson S P, Jeggo P A. DNA double-strand break repair. V(D)J recombination and the involvement of DNA-PK. Trends Biochem. Sci. 20:412-415, 1995.
9. Kasid U, Pfeifer A, Weichselbaum R R, Dritschilo A, and Mark G E: The raf oncogene is associated with a radiation-resistant human laryngeal cancer. Science 237:1039-1041, 1987.
10. Kasid U, Pfeifer A, Brennan T, Beckett M, Weichselbaum R R, Dritschilo A, and Mark G E: Effect of antisense c-raf-1 on tumorigenicity and radiation sensitivity of a human squamous carcinoma. Science 243:1354-1356, 1989.
11. Wolffe, A. P. and Guschin, D., Review: chromatin structural features and targets that regulate transcription, J. Structural Sci., 129, 102-122, 2000.
12. Luo R and Dean D. Chromatin remodeling and transcriptional regulation. JNCI. 91:1288, 1999.
13. Struhl K. Histone acetylation and transcriptional regulatory mechanisms. Genes Dev 12:599-606, 1998.
14. Grunstein M. Histone acetylation in chromatin structure and transcription. Nature 389: 349-352, 1997.
15. Alland L, Muhle R, Hou H, Potes J, Chin L, Schreiber-Agus N, and Depinho R. Role for N-CoR and histone deacetylase in Sin3-mediated transcriptional repression. Nature 387:49, 1997.
16. Brownell J and Allis D. An activity gel assay detects a single, catalyticily active histone acetyltransferase subunit in Tetrahymena macronuclei. Proc Natl Acad Sci USA 92:6364-6368, 1995.
17. Snowden A and Perkins N. Cell cycle regulation of the transcriptional coactivators p300m and CREB binding protein. Biochem Pharmacol 55:1937-1954, 1998.
18. Gu W, Shi X, and Roeder R. Synergistic activation of transcription by CBP and p53. Nature. 387: 819-123, 1997.
19. Hamamori Y, Sartorelli V, Ogryzko V, Puri P, Wu H-Y, Wang J, Nakatani Y, and Kedes L. Regulation of Histone Acetyltransferases p300 and PCAF by the bHLH Protein Twist and Adenoviral Oncoprotein E1A. Cell. 96: 405-413, 1999.
20. Yang W M, Inouye C, Zeng Y Y, Bearss D, and Seto D. Transcriptional repression by YY1 is mediated by interaction with a mammalian homolog of the yeast global regulator RPD3. PNAS USA 83:12845-12850, 1996.
21. Kouzarides T. Histone acetylases and deacetylases in cell proliferation. Curr Opinion Genet Dev 9:40-48, 1999.
22. Taunton J, Hassig C A, and Schreiber S L. A mammalian histone deacetylase related to the yeast transcriptional regulator Rpd3p. Science 272:408-411, 1996.
23. Yang X, Ogysko V, Nishikawa J, Howard B, and Nakatani Y. A p300/CBP-associated factor that competes with the adenoviral oncoprotein E1A. Nature 382:319, 1996.
24. Yang W M, Yao Y L, Sun J M, Davie J R, and Seto E. Isolation and characterization of cDNAs corresponding to an additional member of the human histone deacetylase gene family. J Biol Chem 272:28001-28007, 1997.
25. Emiliani S, Wolfgang F, VanLint C, Al-Abed Y, and Verdin E. Characterization of a human RPD3 ortholog, HDAC3. Proc Natl Acad Sci USA 95:2795-2800, 1998.
26. Kuo M and Allis D. Roles of histone acetyltranferases and deactylases in gene regulation. Bioessays. 20:615-626, 1998.
27. Harper Se, Qiu Y, and Sharp P A. Sin3 corepressor function in Myc-induced transcription and transformation. Proc Natl Acad Sci USA 93:8536, 1996.
28. Zhou X, Richon V M, Rifkind R A, Marks P. Identification of a xcnal repressor related to the noncatalytic domain of histone deacelytones 4 and 5. Proc Natl Acad Sci USA 97:1056-1061, 2000.
29. Bannister A J and Kouzarides T. The CBP co-activator is a histone acetyltransferase. Nature 384:641-643, 1996.
30. Brehn A and Kouzarides T. Retinoblastoma protein meets chromatin. Trends Biochem Sci 24:142-146, 1999.
31. Yarden R I and Brody L C. BRCA1 interacts with components of the histone deacetylase complex. Proc Natl Acad Sci USA 96:4983-4988, 1999.
32. Utley R T, Ikeda K, Grant P, Cote J, Steger D, Eberhater A, John S, and Workamn J. Transcriptional activators direct histone acetyltransferase complexes to nucleosomes. Nature 394:498-502, 1998.
33. Vassilev A, Yamauchi J, Kotain T, Prives C, Avataggiati M, Qin J, and Nakatani Y. The 400 kDa subunit of the PCAF histone acetylase complex belongs to the ATM superfamily. Mol Cell 2:869-876, 1998.
34. Ogryzko V V, Kotani T, Zhang X, Schiltz R, Howard T, Yang X, Howard R, Qin J, and Nakatani Y. Histone-like TAFs within the PCAF histone acetylase complex. Cell 94:35-44, 1998.
35. Siddique H, Zou J, Rao V, and Reddy E. The BRCA2 is a histone acetyltransferase. Oncogene. 16:2283-2285, 1998.
36. Grant P A, Schieltz D, Pray-Grant M G, Yates J R 3rd, and Workman J L. The ATM-related cofactor Tra1 is a component of the purified SAGA complex. Mol Cell 2:863-867, 1998.
37. Mc Mahon S B, Van Buslirk H A, Dugan K A, Copeland T D, and Cole M D. The novel ATM-related protein TRRAP is an essential cofactor for the c-Myc and E2F oncoproteins. Cell 94:363-374, 1998.
38. Render R L, Wang J, and Liu J M. Chromatin remodeling and leukemia: new therapeutic paradigms. Blood 94:417-428, 1999.
39. Brehm A, Miska E, McCance D, Reid J, Bannister A, and Kouarides T. Retinoblastma protein recruits histone deacetylase to repress transcription. Nature 391:597-601, 1998.
40. Magnaghi-Jaulin L. et al. Retinoblastoma protein represses transcription by recruiting a histone deacetylase. Nature 391:601-605, 1998.
41. Ferreira R, Magnaghi-Jaulin L, Robin P, Harel-Bellan A, and Trouche, D. The three members of the pocket proteins family share the ability to repress E2F activity through recruitment of a histone deacetylase. Proc Natl Acad Sci USA 95:10493-10498, 1998.
42. Luo R, Postigo A, and Dean D. Rb interacts with histone deactylase to repress transcription. Cell. 92:463-473, 1998.
43. Herrera R, Chen F, and Weinberg R. Increased histone H1 phosphorylation and relaxed chromatin structure in Rb-deficient fibroblasts. Proc Natl Acad Sci USA. 93:11510-11515, 1996.
44. Kim G D, Choi Y H, Dimtchev A, Jeong S J, Dritschilo A, Jung M. Sensing of ionizing radiation-induced DNA damage by ATM through interaction with histone deacetylase. J Biol Chem, 274:31127-31130, 1999.
45. Weichselbaum R R, Beckett M A, Schwartz J L, Dritschilo A. Radioresistant tumor cells are present in head and neck carcinomas that recur after radiotherapy. Int J Radiat Oncol Biol Phys 75:575-579, 1988.
46. Vindelov L L, Christensen I J, and Nissen N I. A detergent-trypsin method for the preparation of nuclei for flow cytometric DNA analysis. Cytometry 3:322-327, 1983.
47. Adachi N, Kobayashi M, Koyama H. Cell cycle-dependent regulation of the mouse DNA topoisomerase Iia gene promoter. Biochem Biophys Res Commun 230:105-109, 1997.
48. Marks P A, Rifkind R A, Richon V M, Breslow R. Inhibitors of histone deacetylase are potentially effective anticancer agents. Clin Cancer Res. 7:759-760, 2001.
49. Gibaldi M and Perrier D. Pharmacokinetics, 2nd Ed, Marcel Dekker, NY, N.Y., pp. 45-111, 1982.
50. Weichselbaum R R, Hallahan D E, Sukhatme V, Dritschilo A, Sherman M, Kufe D. Biological consequences of gene regulation by ionizing radiation. J. Natl. Cancer Inst. 83:480-484, 1991.
51. Brach M A, Hass R, Sherman M L, Gunji H, Weichselbaum R R, Kufe D. Ionizing radiation induces expression and binding of the nuclear factor kB. J. Clin. Invest. 88:691-695, 1991.
52. Brothwell M. P75NTR. A receptor after all. Science 272:506-507, 1996.
53. Meyn S M, Strasfeld L, Allen C. Testing the role of p53 in genetic instability and apoptosis in ataxia-telangiectasia. Int. J. Radiat. Biol. 66:141-149, 1994.
54. Nicholson D W, All A,. Thornberry N A, Vaillancourt J P, Ding C K, Gallant M, Gareau Y, Griffin P R, Labelle M, Lazebnik Y A, Munday N A, Raju A M, Smulson M E, Yamin T-T, Yu V L, Miller D K. Identification and inhibition of the ICE/CED-3 protease necessary for mammalian apoptosis. Nature 376:37-43, 1995.
55. Yoshida M, Kijima M, Akita M, Beppu T. Potent and specific inhibition of mammalian histone deactylase both in vivo and in vitro by trichostain A. J. Biol. Chem. 265:17174-17179, 1990.
56. Yoshida M, Horinouchi S, Beppu T. Trichostatin A and trapoxin: novel chemical probes for the role of histone acetylation in chromatin structure and function. Bioassays 17:423-430, 1995.
57. Coffey D C, Kutko M C, Glick R D, Butler L M, Heller G, Rifkind R A, Marks P A, Richon V M, La Quaglia M P. The histone deacetylase inhibitor, CBHA, inhibits growth of human neuroblastoma xenografts in vivo, alone and synergistically with all-trans retinoic acid. Cancer Res. 61:3591-3594, 2001.
58. Butler L M, Webb Y, Agus D B, Higgins B, Tolentino T R, Kutko M C, LaQuaglia M P, Drobnjak M, Cordon-Cardo C, Scher H I, Breslow R, Richon V M, Rifid R A, Marks P A. Inhibition of transformed cell growth and induction of cellular differentiation by pyroxamide, an inhibitor of histone deacetylase. Clin Cancer Res. 7:962-970, 2001.
59. Butler L M, Agus D B, Scher H I, Higgins B, Rose A, Cordon-Cardo C, Thaler H T, Rifkind R A, Marks P A, Richon V M. Suberoylanilide hydroxamic acid, an inhibitor of histone deacetylase, suppresses the growth of prostate cancer cells in vitro and in vivo. Cancer Res. 60:5165-5170, 2000.
60. Newmark H L, Lupton J R, Young C W. Butyrate as a differentiating agent: pharmacokinetics, analogues, and current status. Cancer Lett. 78:1-5, 1994.
61. Carducci M, Bowling M K, Eisenberger M, Sinibaldi V, Chen T, Nor D, et al. Phenylbutyrate (PB) for refractory solid tumors: phase I clinical and pharmacologic evaluation of intravenous and oral PB. Anticancer Res. 17:3972-3973, 1997.
62. Tsuji N, Kobayashi M, Nagashima K, Wakisaka Y, Koizumi K. A new antifugal antibiotic, trichostatin. J. Antibiot (Tokyo) 29:1-6, 1976.
63. Cohen B A, Mitra R D, Hughes J D, Church G M. A computational analysis of whole-genome expression data reveals chromosomal domains of gene expression. Nat Genet. 26:183-186, 2000.
64. Kwon H J, Owa T, Hassig C A, Shimada J, Schreiber S L. Depudecin induces morphological reversion of transformed fibroblasts via the inhibition of histone deacetylase. Proc. Natl. Acad. Sci. USA 95: 3356-3361, 1998.
65. Yoshida M, Horinouchi S, and Beppu T. Trichostatin A and trapoxin: novel chemical probes for the role of histon acetylation in chromatin structure and function. Bioessays 17:423-430, 1995.
66. Jung M, Zhang Y, Dimtchev A, Dritschilo A. Impaired regulation of nuclear factor-kB results in apoptosis induced by gamma radiation. Radiat. Res. 149: 596-601, 1998.
67. Banfield J D and Raftery A E. Model-based Gaussian and non-Gaussian clustering. Biomeetrics 49:803-822, 1992.
68. Jolliffe I T. Principal component analysis. New York, Springer-Verlag, 1986.
69. Hosmer D W and Lemeshow S. Applied logistic regression. New York: John Wiley, 1989.
70. Di Paolo A, Danesi R, Caputo S, Macchia M, Lastella M, Boggi U, Mosca F, Marchetti A, Del Tacca M. Inhibition of protein farnesylation enhances the chemotherapeutic efficacy of the novel geranylgeranyltransferase inhibitor BAL9611 in human colon cancer cells. Br J Cancer. 84:1535-43, 2001.
71. Amorino G P, Mohr P J, Hercules S K, Pyo H, Choy H. Combined effects of the orally active cisplatin analog, JM216, and radiation in antitumor therapy. Cancer Chemother Pharmacol. 46:423-6, 2000.
72. Mehta S, Blackinton D, Omar I, Kouttab N, Myrick D, Klostergaard J, Wanebo H. Combined cytotoxic action of paclitaxel and ceramide against the human Tu138 head and neck squamous carcinoma cell line. Cancer Chemother Pharmacol. 46:85-92, 2000.

Each and every reference cited herein is hereby incorporated in its entirety for all purposes to the same extent as if each reference were individually incorporated by reference. Furthermore, while the invention has been described in detail with reference to preferred embodiments thereof, it will be apparent to one skilled in the art that various changes can be made, and equivalents employed, without departing from the scope of the invention.

1. A method for the treatment of cancer comprising,
administering a synergistically effective amount of at least one inhibitor of histone deacetylase; and
administering at least one other anticancer agent selected from the group consisting of radiation, one or more chemotherapeutic agents, and combinations thereof.

2. The method of claim 1, wherein the other anticancer agent is radiation.

3. The method of claim 1, wherein the other anticancer agent is a chemotherapeutic agent.

4. The method of claim 1, wherein the synergistically effective amount provides a concentration of the inhibitor of histone deacetylase in a target tissue about equal to the IC₅₀ of the inhibitor or less.

5. The method of claim 1, wherein the histone deacetylase inhibitor is chosen from among the group of compounds listed in Table I.

6. The method of claim 1, wherein the histone deacetylase inhibitor is selected from the group consisting of trichostatin A, FR, M344, SAHA, and combinations thereof.

7. The method of claim 1, wherein the synergistically effective amount of at least one inhibitor of histone deacetylase is substantially less than an amount of the inhibitor required to promote apoptosis in a target tissue of a host as a single agent.

8. The method of claim 1, wherein the at least one other anticancer agent is selected from the group consisting of cisplatinum, adriamycin (Doxirubicin), topoisomerase inhibitors (Etoposide), 5-FU, taxol and combinations thereof.

9. The method of claim 1, wherein the inhibitor of histone deacetylase is administered directly into a tumor in a host.

10. The method of claim 1, wherein the inhibitor of histone deacetylase is administered locally to a tumor in a host.

11. A method for the treatment of cancer comprising,
administering an effective amount of at least one inhibitor of histone deacetylase in combination with radiation therapy.

12. The method of claim 11, wherein the at least one inhibitor of histone deacetylase is administered for about one or more days prior to the commencement of the radiatation therapy.

13. The method of claim 11, wherein the at least one inhibitor of histone deacetylase is administered about once a day for about 14 days prior to the commencement of the radiation therapy.

14. The method of claim 11, wherein the at least one inhibitor of histone deacetylase is administered on at least about 10 of about 13 days prior to the commencement of the radiation therapy.

15. The method of any of claims 11, 12, 13 or 14, wherein administration of the at least one inhibitor of histone deacetylase continues at least about daily during the entire course of radiation therapy.

16. The method of any one of claims 11, 12, 13 or 14, wherein the amount of at least one inhibitor of histone deacetylase administered is about equal to the IC₅₀ of the inhibitor or less.

17. The method of any one of claims 11, 12, 13 or 14, wherein the amount of at least one inhibitor of histone deacetylase administered is about equal to 50% of the IC₅₀ of the inhibitor or less.

18. The method of any one of claims 11, 12, 13 or 14, wherein the amount of at least one inhibitor of histone deacetylase administered does not cause significant systemic effects.

19. The method of any one of claims 11, 12, 13 or 14, wherein the histone deacetylase inhibitor is chosen from among the group of compounds listed in Table I.

20. The method of any one of claims 11, 12, 13 or 14, wherein the histone deacetylase inhibitor is selected from the group consisting of trichostatin A, FR, M344, SAHA and combinations thereof.

21. A method for treating cancer in a patient in need thereof comprising administering to said patient a first amount of a histone deacetylase inhibitor in a first treatment procedure, and a second amount of radiation in a second treatment procedure wherein, the first and second amounts together comprise a therapeutically effective amount.

22. The method according to claim 21, wherein said HDAC inhibitor is a hydroxamic acid derivative selected from the group consisting of SAHA and Trichostatin A (TSA).

23. The method according to claim 21, wherein said HDAC inhibitor is a Cyclic Tetrapeptide selected from the group consisting of FR901228.

24. The method according to claim 21, wherein said HDAC inhibitor is represented by the following structure: 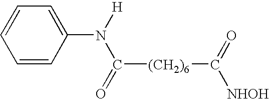
or a pharmaceutically acceptable salt thereof.

25. The method according to claim 21, wherein the radiation of the second treatment procedure is external beam radiation.

26. The method according to claim 21, wherein the radiation of the second treatment procedure is a radiopharmaceutical agent.

27. The method of claim 26, wherein the radiopharmaceutical is a radioactive conjugate.

28. The method according to claim 27, wherein said radioactive conjugate is a radiolabeled antibody.

29. The method according to claim 21, wherein the radiation is selected from the group consisting of: electromagnetic radiation and particulate radiation.

30. The method according to claim 29, wherein the electromagnetic radiation is selected from the group consisting of: x-rays, gamma rays and any combination thereof.

31. The method of claim 29, wherein the particulate radiation is selected from the group consisting of: electron beams (beta particles), protons beams, neutron beams, alpha particles and negative pi mesons.

32. The method of claim 31, wherein the particulate radiation is alpha particles.

33. The method according to claim 21, wherein a total of at least about 1 Gy of radiation is administered to the patient.

34. The method according to claim 21, wherein a total of at least about 10 Gy of radiation is administered to the patient.

35. The method according to claim 21, wherein a total of at least about 20 Gy of radiation is administered to the patient.

36. The method according to claim 21, wherein a total of at least about 40 Gy of radiation is administered to the patient.

37. The method according to claim 21, wherein the therapeutic effect of said HDAC inhibitor and said radiation is synergistic.

38. The method according to claim 36, wherein said HDAC inhibitor sensitizes cancer cells in the patient to radiation.

39. The method according to claim 21, wherein radiation sensitizes cancer cells in the patient to said HDAC inhibitor.

40. The method according to claim 21, wherein said HDAC inhibitor and radiation are administered simultaneously.

41. The method according to claim 21, wherein said HDAC inhibitor and said radiation are administered sequentially.

42. The method according to claim 41, wherein said HDAC inhibitor is administered prior to administering said radiation.

43. The method according to claim 41, wherein said HDAC inhibitor is administered after administering said radiation.

44. The method of claim 21, wherein the HDAC inhibitor is administered orally, parenterally, intraperitoneally, intravenously, intraarterially, transdermally, sublingually, intramuscularly, rectally, transbuccally, intranasally, via inhalation, vaginally, intraoccularly, locally, subcutaneously, intraadiposally, intraarticularly, intrathecally.

45. The method of claim 21, wherein the HDAC inhibitor is in a slow release dosage form.

46. The method of claim 26, wherein the radiopharmaceutical agent is administered orally, parenterally, intraperitoneally, intravenously, intraarterially, transdermally, sublingually, intramuscularly, rectally, transbuccally, intranasally, via inhalation, vaginally, intraoccularly, locally, subcutaneously, intraadiposally, intraarticularly or intrathecally.

47. The method of claim 26, wherein the radiopharmaceutical agent is in a slow release dosage form.

48. A method of determining the sensitivity of a cancer cell to a combination therapy of an HDAC inhibitor and radiation, said method comprising the step of contacting said cancer cell with a first amount of a histone deacetylase inhibitor in a first treatment procedure, and a second amount of radiation in a second treatment procedure, wherein the first and second treatments together comprise a therapeutically effective amount and assessing the sensitivity of the cell to treatment.

49. A method of determining a therapeutically effective amount of a combination of an HDAC inhibitor and radiation for treating a cancer, comprising the step of exposing a cancer cell to a first amount of a histone deacetylase inhibitor in a first treatment procedure, and a second amount or dose of radiation in a second treatment procedure, wherein the first and second treatments together comprise a therapeutically effective amount and assessing the anticancer effects.

50. A pharmaceutical composition comprising a first amount of a histone deacetylase inhibitor and a second amount of radiation wherein the first and second amounts together comprise a therapeutically effective amount.

51. The composition of claim 50, wherein the radiation is a radiopharmaceutical agent.

52. Use of a first amount of an HDAC inhibitor and a second amount of radiation for the manufacture of a medicament for treating cancer.

53. The use of claim 52, wherein the radiation is a radiopharmaceutical agent.
